(19) United States
(12) Patent Application Publication
(10) Pub. No.: US 2024/0298934 A1
(43) Pub. Date: Sep. 12, 2024

(54) DETECTION OF LUNG INFLAMMATION BASED ON GLUCOSE LEVEL IN SPUTUM SAMPLE

(71) Applicants: Mohammad Abdolahad, Tehran (IR); Mohammad Ali Khayamian, Tehran (IR); Mohammadreza Ghaderinia, Tehran (IR); Hamed Abadijoo, Tehran (IR); Hossein simaee, Tehran (IR)

(72) Inventors: Mohammad Abdolahad, Tehran (IR); Mohammad Ali Khayamian, Tehran (IR); Mohammadreza Ghaderinia, Tehran (IR); Hamed Abadijoo, Tehran (IR); Hossein simaee, Tehran (IR)

(73) Assignee: Nano Hesgarsazan Salamat Arya, Tehran, OT (IR)

(21) Appl. No.: 18/666,829

(22) Filed: May 17, 2024

Related U.S. Application Data
(63) Continuation-in-part of application No. PCT/IB2022/051357, filed on Feb. 16, 2022.
(60) Provisional application No. 63/282,231, filed on Nov. 23, 2021.

(51) Int. Cl. A61B 5/145; A61B 5/00; A61B 5/08; A61B 5/1455

(52) U.S. Cl. CPC A61B 5/14532; A61B 5/08; A61B 5/14507; A61B 5/1455; A61B 5/7267

(57) ABSTRACT

A method for detecting and treating lung inflammation. The method includes acquiring a sputum sample from a person suspected to have a lung inflammation, measuring a level of glucose in the sputum sample, detecting lung inflammation status of the person based on the measured level of glucose, and treating the person with a detected lung inflammation by prescribing anti-inflammatory medications to the person based on a clinician's advice. Detecting the lung inflammation status of the person includes detecting no lung inflammation if the measured level of glucose is less than a minimum threshold glucose concentration, or detecting a lung inflammation if the measured level of glucose is equal to or more than a maximum threshold glucose concentration, or detecting a probable lung inflammation if the measured level of glucose is between the minimum threshold glucose concentration and the maximum threshold glucose concentration.

### CROSS-REFERENCE TO RELATED APPLICATION

This application is a continuation-in-part of International Patent Application PCT/IB2022/051357, filed on Feb. 16, 2022, and entitled “DETECTION OF LUNG INFLAMMATION BASED ON GLUCOSE LEVEL IN SPUTUM SAMPLE”, which takes priority from U.S. Provisional Patent Application Ser. No. 63/282,231, filed on Nov. 23, 2021, and entitled “DIAGNOSING LUNG INFLAMMATION IN COVID-19 PATIENTS BY MEASURING GLUCOSE CONCENTRATION IN SPUTUM SAMPLE”, which are both incorporated herein by reference in their entirety.

### TECHNICAL FIELD

The present disclosure generally relates to detection of lung inflammation by analyzing a level of glucose concentration in a sputum sample, and particularly, detecting a lung inflammation in COVID-19 infected patients by measuring level of glucose concentration in their sputum sample.

### BACKGROUND

Lung inflammatory may occur in patients with an inflammatory disease, such as chronic obstructive pulmonary disease (COPD). More specifically, high levels of lung inflammation happens due to an infection with COVID-19 virus. During immunological phase of an inflammatory disease, e.g., COVID-19 infection, cytokine storm may cause dilation of vessels around alveoli in lung and consequently blood may be leaked into air sacs. This inflammatory phase and a probability of lung involvement is currently detected using chest computerized tomography (CT)-scan. However, CT-scan imaging requires complicated and expensive devices and procedures as well as causing many important side effects on patients. Furthermore, CT-scan imaging may not be essential for diagnosis of all cases or at all phases of an inflammatory disease. In addition, in a pandemic phase of an inflammatory disease, such as what happened in case of COVID-19 infection, using an expensive and complex diagnostic system, such as CT-scan imaging is not possible everywhere.

Therefore, there is a need for a rapid, simple, cost-effective, and precise method, system, and associated apparatus for fast and reliable detection of lung inflammation during an inflammatory disease such as COVID-19 infection by analyzing a respiratory system associated sample, such as a sputum sample. Furthermore, there is a need for a fast-diagnosing and easy-to-use method to detect lung inflammation without any need for conducting lung CT-Scan, which requires expensive equipment and also has many important side effects. Moreover, there is a need for a rapid, cost-effective and simple method to detect lung inflammation in COVID-19 patients for early and simple diagnosis of lung infection with COVID-19 as well as avoiding non-necessary CT-scan imaging.

### SUMMARY

This summary is intended to provide an overview of the subject matter of this patent, and is not intended to identify essential elements or key elements of the subject matter, nor is it intended to be used to determine the scope of the claimed embodiments. The proper scope of this patent may be ascertained from the claims set forth below in view of the detailed description below and the drawings.

In one general aspect of the present disclosure, a method for detecting and treating lung inflammation is described. The method may include acquiring a sputum sample from a person suspected to have a lung inflammation, measuring a level of glucose in the sputum sample, detecting, utilizing one or more processors, a lung inflammation for the person if the measured level of glucose in the sputum sample is equal to or more than a maximum threshold glucose concentration by comparing the measured level of glucose in the sputum sample with the maximum threshold glucose concentration, and treating the person with the detected lung inflammation status by prescribing anti-inflammatory medications to the person based on a clinician's advice.

In an exemplary embodiment, detecting the lung inflammation for the person may include detecting the lung inflammation for the person if the measured level of glucose in the sputum sample is 30 mg/dl or more. In an exemplary embodiment, detecting the lung inflammation for the person may include detecting a lung infection with an inflammatory disease for the person. In an exemplary embodiment, detecting the lung infection with the inflammatory disease for the person may include detecting a lung infection with a pulmonary disease for the person. In an exemplary embodiment, detecting the lung infection with the inflammatory disease for the person may include an infection with COVID-19 virus for the person.

In an exemplary embodiment, detecting the lung inflammation status of the person may further include detecting no lung inflammation for the person if the measured level of glucose in the sputum sample is less than a minimum threshold glucose concentration or detecting a probable lung inflammation for the person if the measured level of glucose in the sputum sample is between the minimum threshold glucose concentration and the maximum threshold glucose concentration. In an exemplary embodiment, detecting no lung inflammation for the person may include detecting no lung inflammation for the person if the measured level of glucose in the sputum sample is 10.5 mg/dl or less. In an exemplary embodiment, detecting the probable lung inflammation for the person may include detecting the probable lung inflammation for the person if the measured level of glucose in the sputum sample is between 10.5 mg/dl and 30 mg/dl.

In an exemplary embodiment, detecting the lung inflammation status of the person with the detected probable lung inflammation may further include applying a chest computerized tomography (CT)-scan assay to the person and detecting one of a lung inflammation for the person or no lung inflammation for the person based on an obtained image of the applied CT-scan assay. In an exemplary embodiment, the person with the detected lung inflammation is treated by prescribing anti-inflammatory medications to the person based on a clinician's advice.

In an exemplary embodiment, measuring the level of glucose in the sputum sample may include measuring the level of glucose in the sputum sample by applying a spectrophotometry process to the sputum sample. In an exemplary embodiment, measuring the level of glucose in the sputum sample may include homogenizing the sputum sample, forming a mixture by diluting the homogenized sputum sample in a reagent, putting the sputum sample in interaction with the reagent by maintaining the mixture in an incubator, transmitting a light wave with a wavelength in a range of 300 nm to 400 nm to the incubated mixture, measuring an absorption amount of the transmitted light wave by the incubated mixture, and calculating an amount of glucose in the sputum sample based on the absorption amount of the transmitted light wave by the incubated mixture.

In an exemplary embodiment, homogenizing the sputum sample may include forming a homogeneous and uniform sputum sample fluid by sonicating the sputum sample at a power in a range of 0.5 W to 5 W for a time period in a range of 2 s to 10 s. In an exemplary embodiment, diluting the homogenized sputum sample in the reagent may include mixing 10 μl of the homogenized sputum sample with 1000 μl of an enzymatic spectrophotometric glucose detection reagent. In an exemplary embodiment, maintaining the mixture in the incubator may include maintaining the mixture in the incubator at 37° C. for 10 minutes. In an exemplary embodiment, transmitting the light wave to the incubated mixture and measuring the absorption amount of the transmitted light wave by the incubated mixture are carried out utilizing a spectrophotometer device.

In an exemplary embodiment, the method may further include generating the minimum threshold glucose concentration and the maximum threshold glucose concentration. In an exemplary embodiment, generating the minimum threshold glucose concentration and the maximum threshold glucose concentration may include measuring a set of level of glucose values from a plurality of sputum samples associated with a respective plurality of persons, determining lung inflammation status of the plurality of persons with an inflammatory disease by applying a chest computerized tomography (CT)-scan assay to each respective person, assigning the determined lung inflammation status of each respective person to the measured level of glucose value of the respective sputum sample, classifying the set of level of glucose values into two glucose level ranges based on the determined lung inflammation status assigned to each respective sputum sample of the plurality of persons, and determining the minimum threshold glucose concentration and the maximum threshold glucose concentration based on the two classified glucose level ranges. In an exemplary embodiment, the determined lung inflammation status may include one of no lung inflammation and a lung inflammation based on result of the applied chest CT-scan assay. In an exemplary embodiment, the two glucose level ranges may include a healthy or no lung inflammation range including a first range of level of glucose values assigned as being of the no lung inflammation and a lung inflammation range including a second range of level of glucose values assigned as being of the lung inflammation. In an exemplary embodiment, determining the minimum threshold glucose concentration and the maximum threshold glucose concentration may include determining the maximum threshold glucose concentration equal to a value equal to a minimum level of glucose value among the second range of level of glucose values and a determining the minimum threshold glucose concentration equal to a maximum level of glucose value among values of the first range of level of glucose values.

In another general aspect, the present disclosure describes an exemplary system for detecting lung inflammation. The system may include a spectrophotometer device and a processing unit electrically connected to the spectrophotometer device. In an exemplary embodiment, the spectrophotometer device may be configured to receive a mixture including a sputum sample acquired from a person suspected to have a lung inflammation and measure a level of glucose in the sputum sample. In an exemplary embodiment, the processing unit may include a memory having processor-readable instructions stored therein and a processor. In an exemplary embodiment, the processor may be configured to access the memory and execute the processor-readable instructions. In an exemplary embodiment, executing the processor-readable instructions by the processor may configure the processor to perform a method. The method may include measuring the level of glucose in the sputum sample utilizing the spectrophotometer device, receiving the measured level of glucose in the sputum sample from the spectrophotometer device, and detecting lung inflammation status of the person based on the measured level of glucose in the sputum sample. In an exemplary embodiment, detecting the lung inflammation status of the person may include detecting no lung inflammation if the measured level of glucose in the sputum sample is equal to or less than a minimum threshold glucose concentration, or detecting a lung inflammation if the measured level of glucose in the sputum sample is equal to or more than a maximum threshold glucose concentration, or detecting a probable lung inflammation for the person if the measured level of glucose in the sputum sample is between the minimum threshold glucose concentration and the maximum threshold glucose concentration.

In an exemplary embodiment, detecting the lung inflammation status of the person may include comparing the measured level of glucose in the sputum sample with respective values of the minimum threshold glucose concentration and the maximum threshold glucose concentration. In an exemplary embodiment, the minimum threshold glucose concentration may include a glucose concentration of 10.5 mg/dl. In an exemplary embodiment, the maximum threshold glucose concentration may include a glucose concentration of 30 mg/dl. In an exemplary embodiment, detecting the lung inflammation may include a lung infection with an inflammatory disease. In an exemplary embodiment, the inflammatory disease may include a pulmonary disease. In an exemplary embodiment, the inflammatory disease may include an infection with COVID-19 virus.

In an exemplary embodiment, the method may further include generating the minimum threshold glucose concentration and the maximum threshold glucose concentration. In an exemplary embodiment, generating the minimum threshold glucose concentration and the maximum threshold glucose concentration may include measuring a set of level of glucose values from a plurality of sputum samples associated with a respective plurality of persons, determining lung inflammation status of the plurality of persons with an inflammatory disease by applying a chest computerized tomography (CT)-scan assay to each respective person, assigning the determined lung inflammation status of each respective person to the measured level of glucose value of the respective sputum sample, classifying the set of level of glucose values into two glucose level ranges based on the determined lung inflammation status assigned to each respective sputum sample of the plurality of persons, and determining the minimum threshold glucose concentration and the maximum threshold glucose concentration based on the two classified glucose level ranges. In an exemplary embodiment, the determined lung inflammation status may include one of healthy/no lung inflammation or a lung inflammation based on result of the applied chest CT-scan assay. In an exemplary embodiment, the two glucose level ranges may include a healthy or no lung inflammation range including a first range of level of glucose values assigned as being of the no lung inflammation and a lung inflammation range including a second range of level of glucose values assigned as being of the lung inflammation. In an exemplary embodiment, determining the minimum threshold glucose concentration and the maximum threshold glucose concentration may include determining the maximum threshold glucose concentration equal to a minimum level of glucose value among the second range of level of glucose values and determining the minimum threshold glucose concentration equal to a maximum level of glucose value among values of the first range of level of glucose values.

In an exemplary embodiment, measuring the level of glucose in the sputum sample may include transmitting a light wave with a wavelength in a range of 300 nm to 400 nm to the sputum sample utilizing the spectrophotometer device, measuring an absorption amount of the transmitted light wave by the sputum sample utilizing the spectrophotometer device, and calculating an amount of glucose in the sputum sample based on the absorption amount of the transmitted light wave utilizing the spectrophotometer device.

### BRIEF DESCRIPTION OF THE DRAWINGS

The drawing figures depict one or more embodiments in accord with the present teachings, by way of example only, not by way of limitation. In the figures, like reference numerals refer to the same or similar elements.

FIG. 1 illustrates a schematic view of an exemplary system for detecting lung inflammation, consistent with one or more exemplary embodiments of the present disclosure.

FIG. 2A illustrates an exemplary method for detecting and treating lung inflammation, consistent with one or more exemplary embodiments of the present disclosure.

FIG. 2B illustrates an exemplary method for measuring a level of glucose in an exemplary sputum sample, consistent with one or more exemplary embodiments of the present disclosure.

FIG. 3 illustrates an example computer system in which an embodiment of the present disclosure, or portions thereof, may be implemented as computer-readable code, consistent with one or more exemplary embodiments of the present disclosure.

FIG. 4 illustrates a graph of comparison of measured glucose levels in COVID-19 patients with positive and negative lung CT scans, consistent with one or more exemplary embodiments of the present disclosure.

FIG. 5A illustrates a graph of sputum glucose versus blood glucose of patients in three cohorts of CT positive, CT negative and diabetic, and CT negative and nondiabetic, consistent with one or more exemplary embodiments of the present disclosure.

FIG. 5B illustrates a graph of sputum glucose versus blood glucose of patients in two cohorts of CT positive and diabetic and CT positive and nondiabetic, consistent with one or more exemplary embodiments of the present disclosure.

FIG. 5C illustrates a graph of sputum glucose versus blood glucose of patients in two cohorts of CT negative and diabetic and CT negative and nondiabetic, consistent with one or more exemplary embodiments of the present disclosure.

FIG. 6 shows a graph of receiving operative characteristic (ROC) analysis for the measured glucose levels in COVID-19 patients with positive and negative lung CT scans after exclusion of diabetic cases, consistent with one or more exemplary embodiments of the present disclosure.

### DETAILED DESCRIPTION

In the following detailed description, numerous specific details are set forth by way of examples in order to provide a thorough understanding of the relevant teachings. However, it should be apparent that the present teachings may be practiced without such details. In other instances, well known methods, procedures, components, and/or circuitry have been described at a relatively high-level, without detail, in order to avoid unnecessarily obscuring aspects of the present teachings.

Herein, an exemplary method and system is disclosed for diagnosis of lung inflammatory due to an inflammatory disease, such as an infection with COVID-19 virus. In an exemplary method, a glucose level in a sputum sample of a person suspected to be infected with an inflammatory disease, such as COVID-19 infection may be measured and analyzed as a diagnostic factor for detecting a presence of inflammation in lung tissue caused by the inflammatory disease, such as COVID-19 infection. During immunological phase of an inflammatory disease, e.g., COVID-19 infection, cytokine storm may cause dilation of vessels around alveoli in lung and consequently blood may be leaked into air sacs. This inflammatory phase and a probability of lung involvement is currently detected using chest computerized tomography (CT)-scan. In an exemplary method disclosed herein, glucose level in a sputum sample of a person, for example, a COVID-19 patient may be measured and compared with a reference glucose concentration based on a calibration procedure conducted by CT-scan since a meaningful increase in sputum glucose of patients with positive CT-scan may be detected. Measuring glucose level in sputum sample may be utilized as a cost-effective, simple, and fast diagnostic tool for screening patients with inflammation especially in a medical center where there is no access to a CT-scan machine.

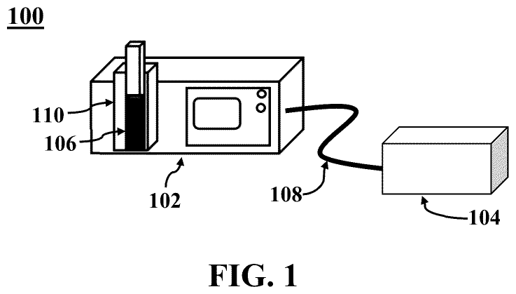

FIG. 1 shows a schematic view of exemplary system 100 for detecting lung inflammation, consistent with one or more exemplary embodiments of the present disclosure. In an exemplary embodiment, system 100 may include spectrophotometer device 102 and processing unit 104 which may be electrically connected to each other via a wireless connection or utilizing an electrically conductive wire 108. In an exemplary embodiment, spectrophotometer device 102 may be configured to receive mixture 106 therein. In an exemplary embodiment, mixture 106 may be placed in an exemplary sample container 110 of spectrophotometer device 102. In an exemplary embodiment, mixture 106 may include a sputum sample acquired from a person suspected to have a lung inflammation. In an exemplary embodiment, spectrophotometer device 102 may be further configured to measure a level of glucose in the sputum sample in mixture 106. In an exemplary embodiment, mixture 106 may include a mixture of an exemplary sputum sample and a reagent. In an exemplary embodiment, the reagent may include a composition or compound which may be used to apply a spectrophotometry process to the sputum sample. In an exemplary embodiment, the reagent may include an enzymatic spectrophotometric glucose detection reagent. In an exemplary embodiment, the reagent may include an enzymatic spectrophotometric glucose detection reagent of a commercial enzymatic spectrophotometric glucose detection kit.

In an exemplary embodiment, mixture 106 may include an exemplary homogenized sputum sample added to the reagent. In an exemplary embodiment, mixture 106 may be maintained in an incubator at 37° C. for about 10 minutes before putting in spectrophotometer device 102. In an exemplary embodiment, an exemplary sputum sample may be homogenized by a sonication process. In an exemplary embodiment, an exemplary sputum sample may be homogenized by sonicating the sputum sample utilizing a sonication device (e.g., a sonication probe) at a power in a rage of about 0.5 W to about 5 W for a time period of 1 s to 10 s. In an exemplary embodiment, an exemplary sputum sample may be homogenized by sonicating the sputum sample utilizing a sonication device at a power of about 1 W for a time period of 2 s to 5 s. In an exemplary embodiment, mixture 106 may include a diluted solution of the sputum sample. In an exemplary embodiment, mixture 106 may include about 10 μl of the homogenized sputum sample added to about 1000 μl of the reagent.

As used herein, “a spectrophotometer device” may refer to a device including a light source and a photodetector. To measure an amount of an analyte in a sample, an exemplary sample may be placed in an exemplary spectrophotometer device similar to spectrophotometer device 102, a light wave with a specific wavelength corresponding to the analyte may be transmitted by the light source to the sample, and an amount of absorbed light may be measured by the photodetector. A concentration of the analyte, e.g., glucose in the sample is determined based on the measured absorbed light according to a calibration dataset including a set of analyte concentrations versus a respective set of amounts of absorbed light.

In an exemplary embodiment, processing unit 104 may include a memory having processor-readable instructions stored therein and a processor. In an exemplary embodiment, the processor may be configured to access the memory and execute the processor-readable instructions. In an exemplary embodiment, when the processor-readable instructions are executed by the processor, configures the processor to perform a method. In an exemplary embodiment, an exemplary method may include one or more steps of an exemplary method for detecting and treating lung inflammation based on measuring a glucose level of an exemplary sputum sample.

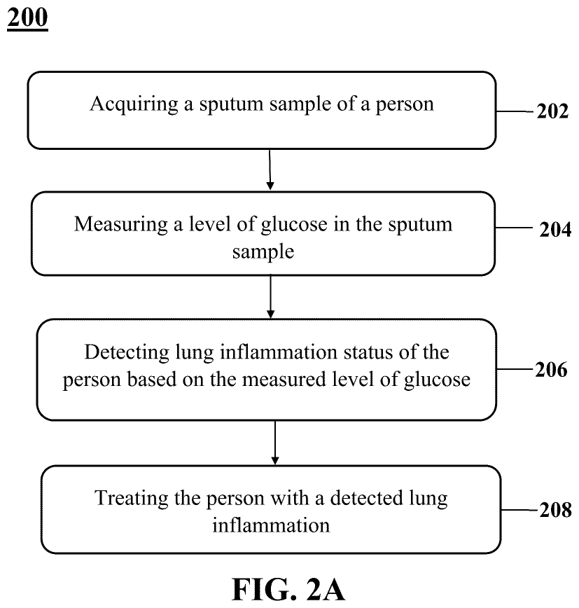

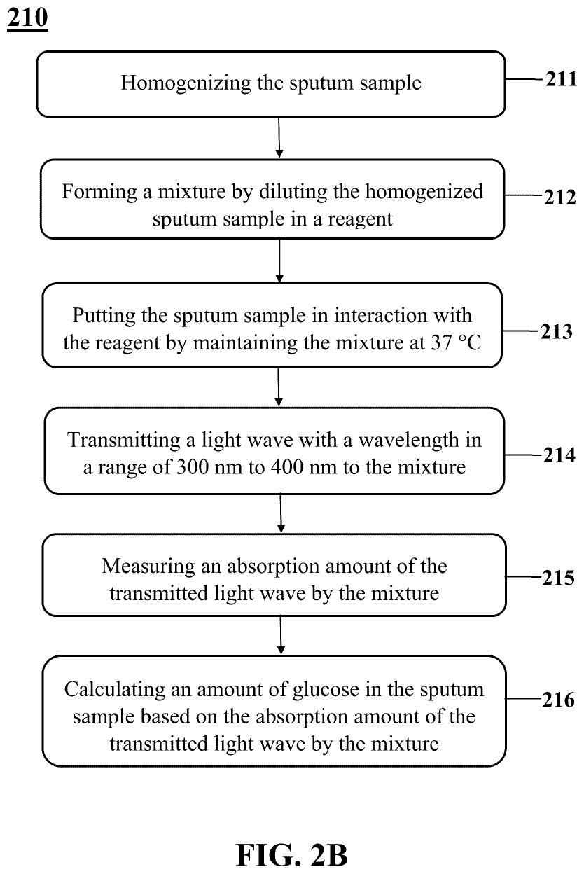

FIG. 2A shows exemplary method 200 for detecting and treating lung inflammation, consistent with one or more exemplary embodiments of the present disclosure. In an exemplary embodiment, one or more steps of exemplary method 200 may be conducted utilizing exemplary system 100. Exemplary method 200 may include acquiring a sputum sample of a person (step 202), measuring a level of glucose in the sputum sample (step 204), detecting lung inflammation status of the person based on the measured level of glucose (step 206), and treating the person with a detected lung inflammation by prescribing anti-inflammatory medications to the person based on a clinician's advice (step 208).

In detail, step 202 may include acquiring a sputum sample of a person. In an exemplary embodiment, step 202 may include acquiring a sputum sample of a person who may be suspected to have a lung infection/inflammation caused by an inflammatory disease. In an exemplary embodiment, the inflammatory disease may be a pulmonary disease. In an exemplary embodiment, the inflammatory disease may include COVID-19 infection.

Furthermore, step 204 may include measuring a level of glucose in the sputum sample. In an exemplary embodiment, measuring the level of glucose in the sputum sample may include measuring the level of glucose in the sputum sample by applying a spectrophotometry process to the sputum sample. In an exemplary embodiment, processing unit 104 may be configured to measure the level of glucose in the sputum sample utilizing spectrophotometer device 102 and receive the measured level of glucose in the sputum sample from spectrophotometer device 102.

FIG. 2B shows a method 210 for measuring a level of glucose in an exemplary sputum sample (step 204), consistent with one or more exemplary embodiments of the present disclosure. In an exemplary embodiment, method 210 may be similar to step 204 of FIG. 2A. In an exemplary embodiment, measuring a level of glucose in an exemplary sputum sample may include homogenizing an exemplary acquired sputum sample (step 211), forming a mixture by diluting the homogenized sputum sample with a reagent (step 212), putting the sputum sample in interaction with the reagent by maintaining the mixture in at 37° C. (step 213), transmitting a light wave with a wavelength in a range of 300 nm to 400 nm to the mixture (step 214), measuring an absorption amount of the transmitted light wave by the mixture (step 215), and calculating an amount of glucose in an exemplary sputum sample based on the absorption amount of the transmitted light wave by the mixture (step 216)

In further detail with respect to step 211, an exemplary acquired sputum sample may be homogenized. In an exemplary embodiment, homogenizing an exemplary acquired sputum sample may include sonicating an exemplary acquired sputum sample utilizing a sonication device. In an exemplary embodiment, homogenizing an exemplary acquired sputum sample may include sonicating an exemplary acquired sputum sample utilizing a sonication device at a power in a range of about 0.5 W to about 5 W for a time period in a range of about 1 seconds to about 10 seconds. In an exemplary embodiment, homogenizing an exemplary acquired sputum sample may include sonicating an exemplary acquired sputum sample utilizing a sonication probe at a power of about 1 W for a time period of about 2 seconds to about 5 seconds.

In further detail with respect to step 212, an exemplary homogenized sputum sample may be diluted in a reagent. In an exemplary embodiment, an exemplary reagent may include may include a spectrophotometric glucose detection reagent, for example, an enzymatic spectrophotometric glucose detection reagent. In an exemplary embodiment, step 212 may include diluting an exemplary homogenized sputum sample with an exemplary reagent by diluting an exemplary homogenized sputum sample with an enzymatic spectrophotometric glucose detection reagent. In an exemplary embodiment, an exemplary reagent may include a reagent material of a glucose detection kit with a detection range of at least 0.23 mg/dl. In an exemplary embodiment, diluting an exemplary homogenized sputum sample with an exemplary reagent may include forming an exemplary mixture by mixing about 10 μl of an exemplary homogenized sputum sample with about 1000 μl of an exemplary reagent. In an exemplary embodiment, an exemplary reagent may include an enzymatic spectrophotometric glucose detection reagent of a commercial enzymatic spectrophotometric glucose detection kit. In an exemplary embodiment, an exemplary formed mixture may be similar to mixture 106 and may be put in spectrophotometer device 102.

In further detail with respect to step 213, an exemplary sputum sample may be put in interaction with an exemplary reagent by maintaining an exemplary mixture at a temperature of about 37° C. In an exemplary embodiment, an exemplary mixture may be kept in an incubator at a temperature of about 37° C.; allowing for reacting an exemplary sputum sample with an exemplary reagent. In an exemplary embodiment, an exemplary mixture may be kept in an incubator at a temperature of about 37° C. for a time period in a range of about 5 minutes to about 30 minutes. In an exemplary embodiment, an exemplary mixture may be kept in an incubator at a temperature of about 37° C. for a time period of about 10 minutes. In an exemplary embodiment, an exemplary incubated mixture may be similar to mixture 106 and may be put in spectrophotometer device 102.

In further detail with respect to step 214, a light wave with a wavelength in a range of about 300 nm to about 400 nm may be transmitted to an exemplary mixture. In an exemplary embodiment, step 214 may include transmitting a light wave with a wavelength in a range of 300 nm to 400 nm to an exemplary incubated mixture. In an exemplary embodiment, step 214 may be carried out utilizing a spectrophotometer device similar to spectrophotometer device 102. In an exemplary embodiment, transmitting a light wave with a wavelength in a range of 300 nm to 400 nm to an exemplary incubated mixture may include transmitting a light wave with a wavelength of about 340 nm to an exemplary incubated mixture utilizing spectrophotometer device 102.

In further detail with respect to step 215, an absorption amount of an exemplary transmitted light wave by an exemplary mixture may be measured. In an exemplary embodiment, step 215 may include measuring an absorption amount of an exemplary transmitted light wave by an exemplary incubated mixture. In an exemplary embodiment, step 215 may be carried out utilizing a spectrophotometer device similar to spectrophotometer device 102.

In further detail with respect to step 216, an amount of glucose in an exemplary sputum sample may be calculated based on an exemplary absorption amount of an exemplary transmitted light wave by an exemplary mixture. In an exemplary embodiment, step 216 may include calculating an amount of glucose in an exemplary sputum sample based on an exemplary absorption amount of an exemplary transmitted light wave by an exemplary incubated mixture. In an exemplary embodiment, calculating an amount of glucose in an exemplary sputum sample (step 216) may include comparing an exemplary absorption amount of an exemplary transmitted light wave by an exemplary incubated mixture with a set of absorption amounts of a calibration dataset associated with a spectrophotometer device similar to spectrophotometer device 102 and detecting an amount of glucose corresponding to an exemplary absorption amount in the calibration dataset.

Moreover, step 206 may include detecting lung inflammation status of the person based on the measured level of glucose. In an exemplary embodiment, detecting the lung inflammation status of the person may include comparing the measured level of glucose in the sputum sample with a threshold glucose concentration range. In an exemplary embodiment, detecting the lung inflammation status of the person may include comparing the measured level of glucose in the sputum sample with two values of a minimum threshold glucose concentration and a maximum threshold glucose concentration. In an exemplary embodiment, detecting the lung inflammation status of the person may include detecting no lung inflammation if the measured level of glucose in the sputum sample is equal to or less than the minimum threshold glucose concentration, or detecting a lung inflammation if the measured level of glucose in the sputum sample is equal to or more than the maximum threshold glucose concentration, or detecting a probable lung inflammation for the person if the measured level of glucose in the sputum sample is between the minimum threshold glucose concentration and the maximum threshold glucose concentration. In an exemplary embodiment, the minimum threshold glucose concentration may include a glucose concentration of about 10.5 milligrams per deciliter (mg/dl) and the maximum threshold glucose concentration may include a glucose concentration of about 30 mg/dl. In an exemplary embodiment, an exemplary lung inflammation status of an exemplary person may be detected to be a probable lung inflammation if the measured level of glucose in the sputum sample is between 10.5 mg/dl and 30 mg/dl.

In a status of detecting a probable lung inflammation for an exemplary person, detecting an exemplary lung inflammation status of an exemplary person may further include applying a chest computerized tomography (CT)-scan assay to an exemplary person and detecting one of a lung inflammation for the person or no lung inflammation for an exemplary person based on an obtained image of an exemplary applied CT-scan assay. In an exemplary embodiment, a definite lung inflammation may be detected for an exemplary person with a detected probable lung inflammation if a lung inflammation is observed in a result (e.g., an image) of an exemplary applied CT-scan assay. In an exemplary embodiment, a definite no lung inflammation may be detected for an exemplary person with a detected probable lung inflammation if no lung inflammation is observed in a result (e.g., an image) of an exemplary applied CT-scan assay.

In detail, step 208 may include treating lung inflammation of an exemplary person with a detected lung inflammation in step 206. In an exemplary embodiment, an exemplary person with a detected lung inflammation may be treated by prescribing anti-inflammatory medications to an exemplary person based on a clinician's advice. In an exemplary embodiment, lung inflammation of an exemplary person with a detected lung inflammation may include providing medications to an exemplary person based on a value of an exemplary measured level of glucose in an exemplary sputum sample and/or other symptoms of an exemplary person.

In an exemplary embodiment, exemplary method 200 may further include generating an exemplary minimum threshold glucose concentration and an exemplary maximum threshold glucose concentration. In an exemplary embodiment, generating an exemplary minimum threshold glucose concentration and an exemplary maximum threshold glucose concentration may include measuring a set of level of glucose values from a plurality of sputum samples associated with a respective plurality of persons, determining lung inflammation status of the plurality of persons with an inflammatory disease by applying a CT-scan assay to each person, assigning the determined lung inflammation status of each person to the measured level of glucose value of the respective sputum sample, classifying the set of level of glucose values into two glucose level ranges based on the determined lung inflammation status assigned to each respective sputum sample of the plurality of persons, and determining an exemplary minimum threshold glucose concentration and an exemplary maximum threshold glucose concentration based on the two classified glucose level ranges. In an exemplary embodiment, measuring an exemplary set of level of glucose values from an exemplary plurality of sputum samples associated with an exemplary respective plurality of persons may be carried out utilizing a spectrophotometry procedure. In an exemplary embodiment, measuring an exemplary set of level of glucose values from an exemplary plurality of sputum samples associated with an exemplary respective plurality of persons may be carried out utilizing a method similar to method 210 described hereinabove. In an exemplary embodiment, an exemplary determined lung inflammation status may include one of no lung inflammation or a lung inflammation based on result of the applied chest CT-scan assay. In an exemplary embodiment, a long inflammation may be determined for a person if an inflammation is observed in an image obtained from an exemplary applied chest CT-scan assay. In an exemplary embodiment, a status of no long inflammation may be determined for a person if no inflammation is observed in an image obtained from an exemplary applied chest CT-scan assay. In an exemplary embodiment, exemplary two glucose level ranges may include a healthy or no lung inflammation range including a first range of level of glucose values assigned as being of the no lung inflammation and a lung inflammation range including a second range of level of glucose values assigned as being of the lung inflammation. In an exemplary embodiment, determining an exemplary minimum threshold glucose concentration and an exemplary maximum threshold glucose concentration may include determining an exemplary maximum threshold glucose concentration equal to a minimum level of glucose value among an exemplary second range of level of glucose values and determining a minimum threshold glucose concentration equal to a maximum level of glucose value among values of an exemplary first range of level of glucose values. In an exemplary embodiment, an exemplary minimum threshold glucose concentration may be determined equal to 10.5 mg/dl. In an exemplary embodiment, an exemplary maximum threshold glucose concentration may be determined equal to 30 mg/dl.

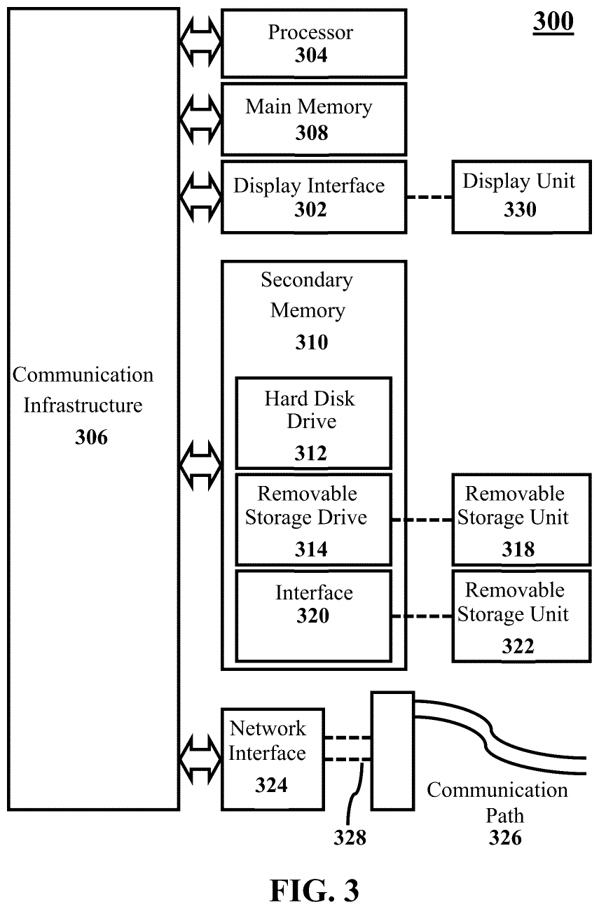

FIG. 3 shows an example computer system 300 in which an embodiment of the present disclosure, or portions thereof, may be implemented as computer-readable code, consistent with one or more exemplary embodiments of the present disclosure. For example, computer system 300 may include an example of processing unit 104, and steps 204 and 206 of flowchart presented in FIG. 2A as well as steps 215 and 216 of flowchart presented in FIG. 2B, may be implemented in computer system 300 using hardware, software, firmware, tangible computer readable media having instructions stored thereon, or a combination thereof and may be implemented in one or more computer systems or other processing systems. Hardware, software, or any combination of such may embody any of the modules and components in FIGS. 1, 2A and 2B.

If programmable logic is used, such logic may execute on a commercially available processing platform or a special purpose device. One ordinary skill in the art may appreciate that an embodiment of the disclosed subject matter can be practiced with various computer system configurations, including multi-core multiprocessor systems, minicomputers, mainframe computers, computers linked or clustered with distributed functions, as well as pervasive or miniature computers that may be embedded into virtually any device.

For instance, a computing device having at least one processor device and a memory may be used to implement the above-described embodiments. A processor device may be a single processor, a plurality of processors, or combinations thereof. Processor devices may have one or more processor “cores”.

An embodiment of the present disclosure is described in terms of this example computer system 300. After reading this description, it will become apparent to a person skilled in the relevant art how to implement the invention using other computer systems and/or computer architectures. Although operations may be described as a sequential process, some of the operations may in fact be performed in parallel, concurrently, and/or in a distributed environment, and with program code stored locally or remotely for access by single or multi-processor machines. In addition, in some embodiments the order of operations may be rearranged without departing from the spirit of the disclosed subject matter.

Processor device 304 may be a special purpose or a general-purpose processor device. As will be appreciated by persons skilled in the relevant art, processor device 304 may also be a single processor in a multi-core/multiprocessor system, such system operating alone, or in a cluster of computing devices operating in a cluster or server farm. Processor device 304 may be connected to a communication infrastructure 306, for example, a bus, message queue, network, or multi-core message-passing scheme.

In an exemplary embodiment, computer system 300 may include a display interface 302, for example a video connector, to transfer data to a display unit 330, for example, a monitor. Computer system 300 may also include a main memory 308, for example, random access memory (RAM), and may also include a secondary memory 310. Secondary memory 310 may include, for example, a hard disk drive 312, and a removable storage drive 314. Removable storage drive 314 may include a floppy disk drive, a magnetic tape drive, an optical disk drive, a flash memory, or the like. Removable storage drive 314 may read from and/or write to a removable storage unit 318 in a well-known manner. Removable storage unit 318 may include a floppy disk, a magnetic tape, an optical disk, etc., which may be read by and written to by removable storage drive 314. As will be appreciated by persons skilled in the relevant art, removable storage unit 318 may include a computer usable storage medium having stored therein computer software and/or data.

In alternative embodiments, secondary memory 310 may include other similar means for allowing computer programs or other instructions to be loaded into computer system 300. Such means may include, for example, a removable storage unit 322 and an interface 320. Examples of such means may include a program cartridge and cartridge interface (such as that found in video game devices), a removable memory chip (such as an EPROM, or PROM) and associated socket, and other removable storage units 322 and interfaces 320 which allow software and data to be transferred from removable storage unit 322 to computer system 300.

Computer system 300 may also include a communications interface 324. Communications interface 324 allows software and data to be transferred between computer system 300 and external devices. Communications interface 324 may include a modem, a network interface (such as an Ethernet card), a communications port, a PCMCIA slot and card, or the like. Software and data transferred via communications interface 324 may be in the form of signals, which may be electronic, electromagnetic, optical, or other signals capable of being received by communications interface 324. These signals may be provided to communications interface 324 via a communications path 326. Communications path 326 carries signals and may be implemented using wire or cable, fiber optics, a phone line, a cellular phone link, an RF link or other communications channels.

In this document, the terms “computer program medium” and “computer usable medium” are used to generally refer to media such as removable storage unit 318, removable storage unit 322, and a hard disk installed in hard disk drive 312. Computer program medium and computer usable medium may also refer to memories, such as main memory 308 and secondary memory 310, which may be memory semiconductors (e.g. DRAMs, etc.).

Computer programs (also called computer control logic) are stored in main memory 308 and/or secondary memory 310. Computer programs may also be received via communications interface 324. Such computer programs, when executed, enable computer system 300 to implement different embodiments of the present disclosure as discussed herein. In particular, the computer programs, when executed, enable processor device 304 to implement the processes of the present disclosure, such as the operations in methods 200 and 210 illustrated by FIGS. 2A and 2B, discussed above. Accordingly, such computer programs represent controllers of computer system 300. Where an exemplary embodiment of method 200 is implemented using software, the software may be stored in a computer program product and loaded into computer system 300 using removable storage drive 314, interface 320, and hard disk drive 312, or communications interface 324.

Embodiments of the present disclosure also may be directed to computer program products including software stored on any computer useable medium. Such software, when executed in one or more data processing device, causes a data processing device to operate as described herein. An embodiment of the present disclosure may employ any computer useable or readable medium. Examples of computer useable mediums include, but are not limited to, primary storage devices (e.g., any type of random access memory), secondary storage devices (e.g., hard drives, floppy disks, CD ROMS, ZIP disks, tapes, magnetic storage devices, and optical storage devices, MEMS, nanotechnological storage device, etc.).

### Example 1: Detection of Lung Inflammation Status by Measuring Glucose Amount in Sputum Samples

In this example, an exemplary system similar to system 100 was utilized to conduct an exemplary method similar to methods 200 and 210 described hereinabove for measuring glucose levels of sputum samples acquired from people suspicious to be infected with COVID-19 virus and diagnosing COVID-19 infection of them based on the measured glucose levels. The measured glucose levels of sputum samples were analyzed by comparing with results of CT-scan data obtained by applying chest CT-scan to the suspected people. An effect of diabetes was also assessed to exclude an effect of blood glucose on sputum glucose concentration. Glucose levels of sputum samples with similar volumes for all people were measured by an enzymatic spectrophotometric glucose detection approach utilizing a spectrophotometer device similar to spectrophotometer device 102. Sputum samples were acquired of 200 participants. All the participants were chosen from non-alcoholic and non-smoker people, and they were asked not to eat sugar containing foods one hour prior to the test. CT-Scan results validated a presence of lung inflammation. For measuring sputum glucose, spectroscopy was utilized as a promising method to identify highly specific vibrational modes of glucose molecules with very high sensitivity. Accordingly, 10 μL of each sputum sample was diluted in 1000 μL of a reagent and the obtained mixture was stored in an incubator at 37° C. for 10 min. Consequently, a spectrophotometer was used to measure the glucose concentration in each sputum sample by measuring an amount of light absorption by the diluted sputum samples.

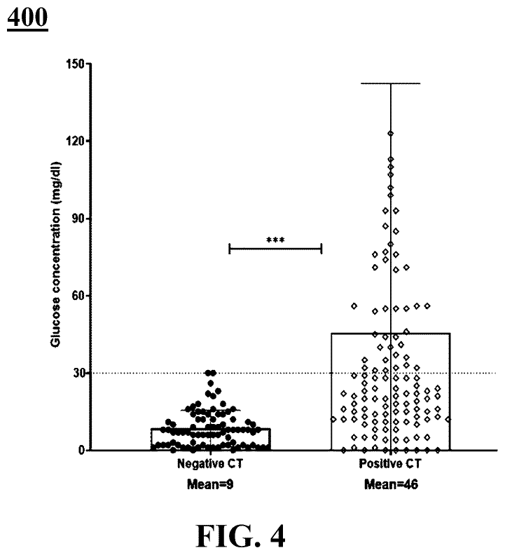

Results of sputum glucose levels (SGL) distribution were compared with the cases with positive and negative CT-Scan results. FIG. 4 shows a graph 400 of comparison of measured glucose levels in COVID-19 patients with positive and negative lung CT scans, consistent with one or more exemplary embodiments of the present disclosure. There is a meaningful discrepancy between the SGL of the negative and positive cases. It is observable that minimum and maximum SGL of the cases with negative CT-Scan were 0 and 30, respectively, while these values in cases with positive SGL were 0 and 120. Moreover, an increased shift in average SGL value of cases with positive CT-Scan could be observed with respect to negative ones. An average of the SGL value in negative cases is about 9 while for positive cases it is about 49. This shows that a rationale threshold specification could be utilized as a diagnostic parameter for detecting lung inflammation in COVID-19 patients with positive CT scan from their sputum glucose level.

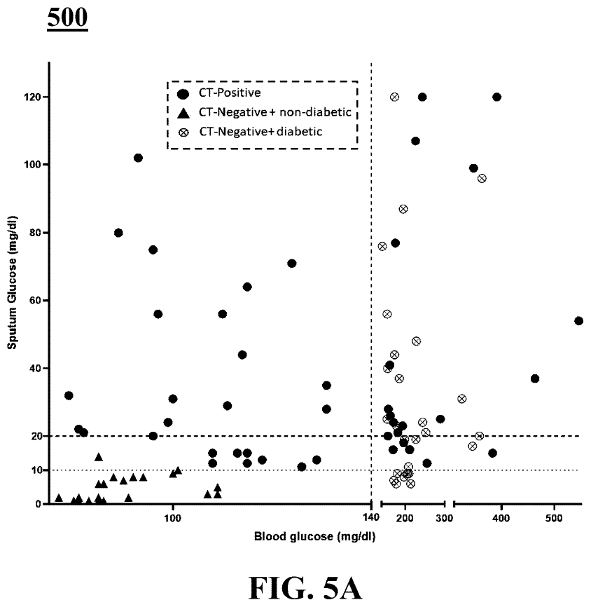

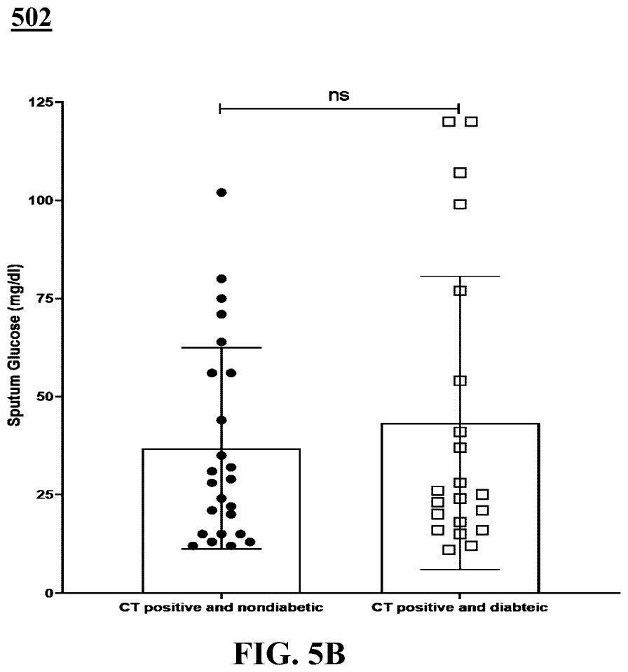

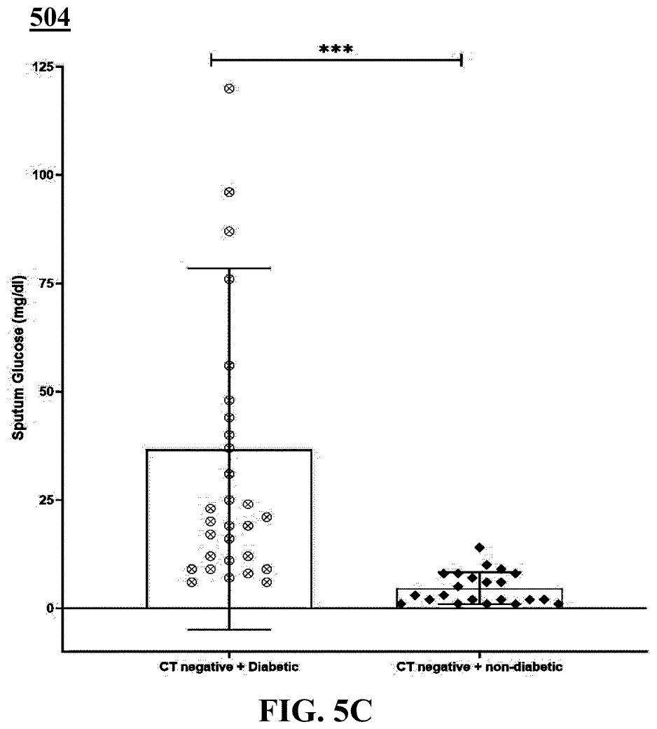

To assess possible effect of blood glucose on concentration of sputum glucose, blood sugar (BS) of patients was tested to distinct diabetic from non-diabetic cases and compare their SGL with their BS. FIG. 5A shows a graph 500 of sputum glucose versus blood glucose of patients in three cohorts of CT positive, CT negative and diabetic, and CT negative and nondiabetic, consistent with one or more exemplary embodiments of the present disclosure. A 140 mg/dl limit was considered as a threshold for distinguishing between diabetic and nondiabetic cases. FIG. 5B shows a graph 502 of sputum glucose versus blood glucose of patients in two cohorts of CT positive and diabetic and CT positive and nondiabetic, consistent with one or more exemplary embodiments of the present disclosure. As could be deduced from FIGS. 5A and 5B, blood glucose of all of CT positive patients spans in both diabetic range (BS of more than about 140 mg/dl) and nondiabetic range (BS of less than about 140 mg/dl) without any meanningful difference. To unveil an effect of the BS on the SGL, two more cohorts were added and analyzed. Sputum samples from healthy donors with clear CT scan but in two groups of diabetic and nondiabetic were collected and their SGL was measured. FIG. 5C shows a graph 504 of sputum glucose versus blood glucose of patients in two cohorts of CT negative and diabetic and CT negative and nondiabetic, consistent with one or more exemplary embodiments of the present disclosure. As presented in FIG. 5C, a meaningful increase in sputum glucose levels of diabetic cases with negative CT with an average of 37 mg/dl of sputum glucose level could be observed compared to nondiabetic cases with negative CT with an average of 5 mg/dl of sputum glucose level. Also, data in diabetic cases are dispersed between 6 to 120 mg/dl while data variation in nondiabetic cases is very small and respective values span from 1 to 14 mg/dl. Such a finding is due to passive diffusion of the serum biomolecules such as hormones, electrolytes etc. from blood capillaries to the salivary glands and is reported previously elsewhere. The results reveal that diabetic cases should be excluded from results and just sputum samples of nondiabetic cases should be considered for detecting lung inflammation based on a measured level of glucose in a sputum sample of a person.

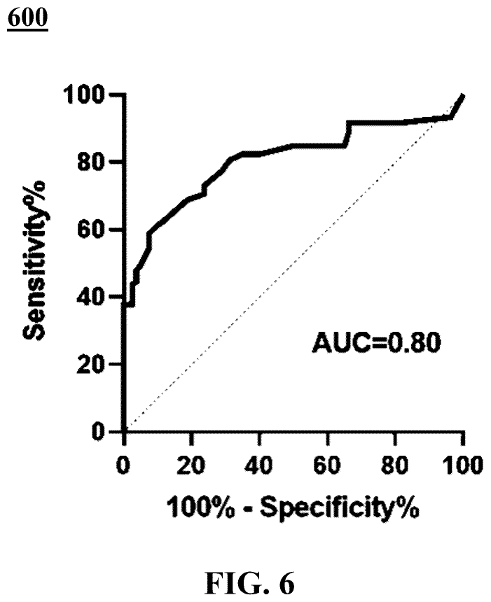

Accordingly, presence of diabetes in a patient may induce perturbation in COVID-19 related SGL results and independently elevated the SGL. Hence, patients with diabetics should be excluded from an analysis of sputum glucose for detection of lung inflammation status. FIG. 6 shows a graph 600 of receiving operative characteristic (ROC) analysis for the measured glucose levels in COVID-19 patients with positive and negative lung CT scans after exclusion of diabetic cases, consistent with one or more exemplary embodiments of the present disclosure. By excluding the diabetic cases and plotting the ROC analysis, an area under the ROC curve (AUC) of 0.80 was obtained and by considering the SGL of 10.5, the best sensitivity of about 81% and specificity of about 70% was reached.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments. This is for purposes of streamlining the disclosure, and is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

While various embodiments have been described, the description is intended to be exemplary, rather than limiting and it will be apparent to those of ordinary skill in the art that many more embodiments and embodiments are possible that are within the scope of the embodiments. Although many possible combinations of features are shown in the accompanying figures and discussed in this detailed description, many other combinations of the disclosed features are possible. Any feature of any embodiment may be used in combination with or substituted for any other feature or element in any other embodiment unless specifically restricted. Therefore, it will be understood that any of the features shown and/or discussed in the present disclosure may be implemented together in any suitable combination. Accordingly, the embodiments are not to be restricted except in light of the attached claims and their equivalents. Also, various modifications and changes may be made within the scope of the attached claims.

What is claimed is:

1. A method for detecting and treating lung inflammation, comprising:
acquiring a sputum sample from a person suspected to have a lung inflammation;
measuring a level of glucose in the sputum sample by applying a spectrophotometry process to the sputum sample, measuring the level of glucose in the sputum sample comprising:
homogenizing the sputum sample by sonicating the sputum sample at a power of 1 W for a time period in a range of 2 s to 5 s;
forming a mixture by diluting 10 μl of the homogenized sputum sample in 1000 μl of a spectrophotometric glucose detection reagent;
putting the homogenized sputum sample in interaction with the spectrophotometric glucose detection reagent by incubating the mixture at 37° C. for 10 minutes;
transmitting a light wave with a wavelength in a range of 300 nm to 400 nm to the incubated mixture utilizing a spectrophotometer device;
measuring an absorption amount of the transmitted light wave by the incubated mixture utilizing the spectrophotometer device; and
calculating an amount of glucose in the sputum sample based on the absorption amount of the transmitted light wave by the incubated mixture;
detecting, utilizing one or more processors, a lung inflammation status of the person, comprising:
detecting no lung inflammation for the person if the measured level of glucose in the sputum sample is less than a minimum threshold glucose concentration of 10.5 mg/dl; or
detecting a probable lung inflammation if the measured level of glucose in the sputum sample is between the minimum threshold glucose concentration of 10.5 mg/dl and a maximum threshold glucose concentration of 30 mg/dl; or
detecting a lung inflammation for the person if the measured level of glucose in the sputum sample is equal to or more than the maximum threshold glucose concentration of 30 mg/dl; and
treating the person with the detected lung inflammation status by prescribing anti-inflammatory medications to the person based on a clinician's advice.

2. The method of claim 1, wherein detecting the lung inflammation status of the person with the detected probable lung inflammation further comprises:
applying a chest computerized tomography (CT)-scan assay to the person; and
detecting one of a lung inflammation for the person or no lung inflammation for the person based on an obtained image of the applied CT-scan assay.

3. The method of claim 2, wherein the person with the detected lung inflammation is treated by prescribing anti-inflammatory medications to the person based on a clinician's advice.

4. A method for detecting and treating lung inflammation, comprising:
acquiring a sputum sample from a person suspected to have a lung inflammation;
measuring a level of glucose in the sputum sample by applying a spectrophotometry process to the sputum sample, measuring the level of glucose in the sputum sample comprising:
homogenizing the sputum sample;
forming a mixture by diluting the homogenized sputum sample in a reagent;
maintaining the mixture in an incubator;
transmitting a light wave with a wavelength in a range of 300 nm to 400 nm to the incubated mixture;
measuring an absorption amount of the transmitted light wave by the incubated mixture; and
calculating an amount of glucose in the sputum sample based on the absorption amount of the transmitted light wave by the incubated mixture;
detecting, utilizing one or more processors, a lung inflammation status of the person, comprising detecting a lung inflammation for the person if the measured level of glucose in the sputum sample is equal to or more than a maximum threshold glucose concentration by comparing the measured level of glucose in the sputum sample with the maximum threshold glucose concentration; and
treating the person with the detected lung inflammation by prescribing anti-inflammatory medications to the person based on a clinician's advice.

5. The method of claim 4, wherein detecting the lung inflammation for the person comprises detecting the lung inflammation for the person if the measured level of glucose in the sputum sample is 30 mg/dl or more.

6. The method of claim 4, wherein detecting the lung inflammation comprises detecting a lung infection with an inflammatory disease.

7. The method of claim 6, wherein detecting the lung infection with the inflammatory disease comprises detecting a lung infection with a pulmonary disease.

8. The method of claim 6, wherein detecting the lung infection with the inflammatory disease comprises detecting an infection with COVID-19 virus.

9. The method of claim 4, wherein detecting the lung inflammation status of the person further comprises:
detecting no lung inflammation for the person if the measured level of glucose in the sputum sample is less than a minimum threshold glucose concentration; or
detecting a probable lung inflammation for the person if the measured level of glucose in the sputum sample is between the minimum threshold glucose concentration and the maximum threshold glucose concentration.

10. The method of claim 9, wherein detecting no lung inflammation for the person comprises detecting no lung inflammation for the person if the measured level of glucose in the sputum sample is 10.5 mg/dl or less.

11. The method of claim 9, wherein detecting the probable lung inflammation for the person comprises detecting the probable lung inflammation for the person if the measured level of glucose in the sputum sample is between 10.5 mg/dl and 30 mg/dl.

12. The method of claim 9, wherein detecting the lung inflammation status of the person with the detected probable lung inflammation further comprises:
applying a chest computerized tomography (CT)-scan assay to the person; and
detecting one of a lung inflammation for the person or no lung inflammation for the person based on an obtained image of the applied CT-scan assay.

13. The method of claim 12, wherein the person with the detected lung inflammation is treated by prescribing anti-inflammatory medications to the person based on a clinician's advice.

14. The method of claim 4, further comprising generating the minimum threshold glucose concentration and the maximum threshold glucose concentration, comprising:
measuring a set of level of glucose values from a plurality of sputum samples associated with a respective plurality of persons;
determining lung inflammation status of the plurality of persons with an inflammatory disease by applying a chest computerized tomography (CT)-scan assay to each respective person, the determined lung inflammation status comprising one of no lung inflammation or a lung inflammation, based on result of the applied chest CT-scan assay;
assigning the determined lung inflammation status of each respective person to the measured level of glucose value of the respective sputum sample;
classifying the set of level of glucose values into two glucose level ranges based on the determined lung inflammation status assigned to each respective sputum sample of the plurality of persons, the two glucose level ranges comprising:
a healthy/no lung inflammation range comprising a first range of level of glucose values assigned as being of the no lung inflammation; and
a lung inflammation range comprising a second range of level of glucose values assigned as being of the lung inflammation; and
determining the maximum threshold glucose concentration equal to a minimum level of glucose value among the second range of level of glucose values and the minimum threshold glucose concentration equal to a maximum level of glucose value among values of the first range of level of glucose values.

15. The method of claim 4, wherein homogenizing the sputum sample comprises forming a homogeneous and uniform sputum sample fluid by sonicating the sputum sample at a power in a range of 0.5 W to 5 W for a time period in a range of 2 s to 10 s.

16. The method of claim 4, wherein diluting the homogenized sputum sample in the reagent comprises mixing 10 μl of the homogenized sputum sample with 1000 μl of an enzymatic spectrophotometric glucose detection reagent.

17. The method of claim 4, wherein maintaining the mixture in the incubator comprises maintaining the mixture in the incubator at 37° C. for 10 minutes.

18. The method of claim 4, wherein transmitting the light wave to the incubated mixture and measuring the absorption amount of the transmitted light wave by the incubated mixture are carried out utilizing a spectrophotometer device.
